(19) United States
(12) Patent Application Publication
(10) Pub. No.: US 2012/0245422 A1
(43) Pub. Date: Sep. 27, 2012

(54) OTOSCOPE ATTACHMENT TO BE USED IN CONJUNCTION WITH A SMART PHONE AND A METHOD FOR ITS USE

(76) Inventor: William M. Hasbun, Mount Laurel, NJ (US)

(21) Appl. No.: 13/052,890

(22) Filed: Mar. 21, 2011

(51) Int. Cl. A61B 1/227

(52) U.S. Cl. 600/200

(57) ABSTRACT

The present otoscope attachment, when used with a cellular phone comprising a light source, provides a powerful otoscope that is both lightweight and portable. This otoscope is powered by one or more light sources comprising the cellular phone, which provides the light necessary to conduct a medical examination with the otoscope. A further feature of this device is its ability to be folded flat making it easy to carry in one's pocket.

### FIELD OF THE INVENTION

The present device relates to diagnostic equipment for use by medical professionals. More particularly it relates to a removable attachment, which when used with a smart phone, creates a functioning otoscope capable of performing medical diagnostic procedures. Smart phones are cellular phones that comprise built-in computers and a camera among other features. The present device incorporates the use of one or more powerful light sources, now commonly found on smart phones, to generate sufficient light to power the otoscope attachment.

### BACKGROUND

Otoscopes are commonly used by medical professionals to observe dark, recessed areas of the body such as the sinuses and the inner ear. Otoscopes typically comprise a hollow conical piece, called a speculum, capable of holding open nostrils and ear canals. The otoscope allows the user to see through this hollow speculum as well as to shine light through it. Most otoscopes also comprise one or more magnifying lenses, allowing the user an even greater ability to view the sinuses and the inner ear. As many common illnesses and infections present visible symptoms in the inner nose and the inner ear, an otoscope is an indispensable diagnostic tool for medical professionals.

A typical otoscope comprises a handle, which houses one or more rechargeable batteries, a light source, a speculum and a lens section. Such otoscopes are commonly found in exam rooms where they are kept in battery-recharging bases. These are not large devices, typically no larger than a medium-sized flashlight, however, they are too large and too heavy to be carried easily in one's pocket. As with nearly all devices, convenience is improved when the otoscope is made smaller and lighter. This is particularly true for doctors and other medical professionals who must carry numerous pieces of medical equipment at one time in addition to smart phones and pagers.

What is needed is an otoscope attachment, to be used in conjunction with powerful light sources commonly found on many smart phones. Furthermore, when in storage, the otoscope attached to the phone must be capable of fitting easily and comfortably inside of a pocket, or capable of easily being carried externally on a person's body.

### SUMMARY OF THE INVENTION

It is an aspect of the present device to provide an otoscope attachment, which can be powered by at least one light source comprising a smart phone, wherein the otoscope attachment is capable of being folded nearly flat when not in use, and place in a shirt or coat pocket.

The above aspect can be obtained by an otoscope attachment for use with a smart phone comprising a phone cover configured to removably connect to the back of a smart phone, wherein the smart phone comprises one or more light sources a working section comprising a speculum assembly and a lens assembly, the speculum assembly further comprising a light pipe configured to transmit light from the light source through a speculum removably connected to the speculum assembly.

The above aspect can also be obtained by an otoscope attachment for use with a smart phone having at least one light source comprising a phone cover configured to removably connect to the back of a smart phone, a working section configured to removably connect to the phone cover, the working section further comprising a speculum assembly and a lens assembly, wherein the speculum assembly comprises a light pipe configured to transmit light from the light source through a speculum removably connected to the speculum assembly, and the working section can be folded nearly flat and reconnected to the phone cover.

The above aspect can also be obtained by a method for using an otoscope attachment, the method comprising providing a smart phone having at least one light source, a speculum, and an otoscope attachment for use with a smart phone having at least one light source comprising a phone cover configured to removably connect to the back of a smart phone, a working section configured to removably connect to the phone cover, the working section further comprising a speculum assembly and a lens assembly, wherein the speculum assembly comprises a light pipe configured to transmit light from the light source through a speculum removably connected to the speculum assembly, and wherein the working section can be folded nearly flat and reconnected to the phone cover, removably connecting the phone cover to the back of the smart phone such that the light source can shine light through a hole in the phone cover, connecting a speculum to the speculum assembly, connecting the working section to the phone cover such that the light source can shine light through the light pipe comprising the speculum assembly, through the speculum, positioning the lens assembly so that a clear line of sight exists from the users eye, through the lens and through the speculum, and using this smart phone and otoscope attachment to examine a patient.

### BRIEF DESCRIPTION OF THE DRAWINGS

Further features and advantages of the present device, as well as the structure and operation of various embodiments of the present device, will become apparent and more readily appreciated from the following description of the preferred embodiments, taken in conjunction with the accompanying drawings of which:

FIG. 1/8 is a front and side perspective view of a typical smart phone comprising a camera and one or more light sources;

FIG. 2/8 is a front and side perspective view of an otoscope attachment connected to a smart phone, according to an embodiment;

FIG. 3/8 is a front and side perspective view of a smart phone connected to an otoscope attachment, according to an embodiment;

FIG. 4/8 is a rear view of an otoscope attachment in an open configuration, according to an embodiment;

FIG. 5/8 is an exploded side perspective view of an otoscope attachment, according to an embodiment

FIG. 6/8 is a front and side perspective view of an otoscope attachment in an open configuration, according to an embodiment;

FIG. 7/8 is a side perspective view of an otoscope attachment in an open configuration, according to an embodiment; and

FIG. 8/8 is a side perspective view of an otoscope attachment in a closed configuration, according to an embodiment.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

Reference will now be made in detail to the presently preferred embodiments of the invention, examples of which are illustrated in the accompanying drawings, wherein like reference numerals refer to like elements throughout.

Many of today's commonly used smart phones comprise cameras on the side opposite the phone's face, and many also comprise one or more powerful light source(s), which can be used as a flash for the camera. The light source can be, for example, a light emitting diode (LED), LED lamp, light bulb, or any other light source that can be embedded into a cellular phone. Some of these phones allow the user to control these light sources and use them as a flashlight. The current device takes advantage of this feature to create a portable and powerful otoscope.

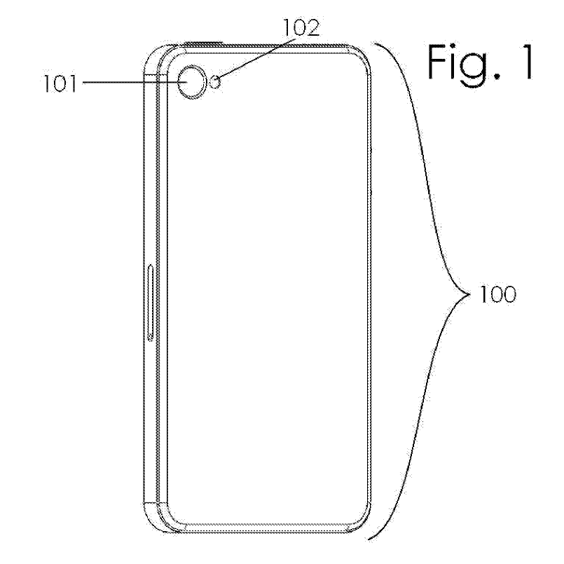

FIG. 1/8 is a rear perspective view of a typical smart phone 100 comprising a camera 101 and one or more light source 102.

Although the present oto scope attachment works best with a smart phone 100 having its camera 101 and light source 102 located in the upper left-hand corner of the back of a smart phone, it could also be configured to work with light sources 102 located at different locations on the phone. Typically, the light source(s) 102 should be located near an outer edge of the phone.

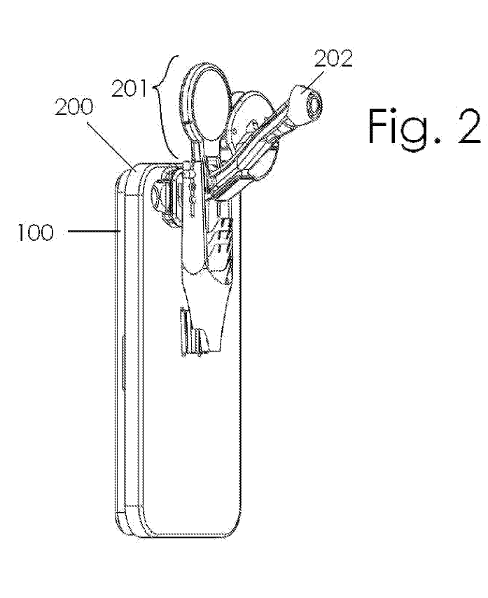

FIG. 2/8 is a front and side perspective view of an otoscope attachment 200 in its open configuration connected to a smart phone 100, according to an embodiment.

In this figure, the location of the lens apparatus 201 and the specula apparatus 202 comprising the otoscope attachment 200 can be seen in relation to the smart phone 100. Specifically, the lens apparatus 201 must be located above the light source 102 and be clear of the smart phone 100 in order to maintain an unobstructed line of sight from the user's eye through the lens and through the speculum.

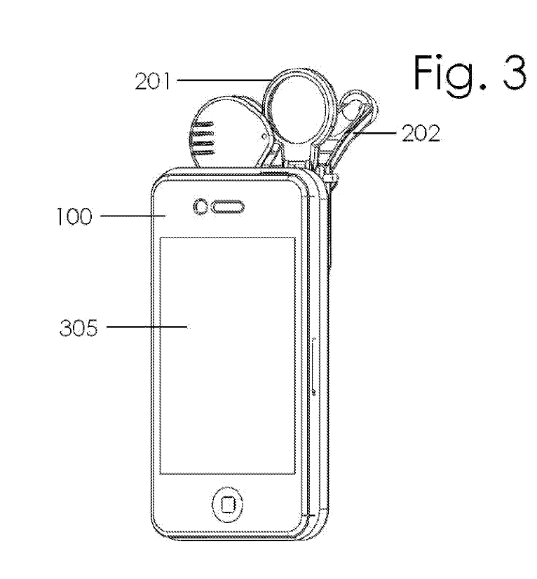

FIG. 3/8 is a rear and side perspective view of an otoscope attachment 200 in its open configuration connected to a smart phone 100, according to an embodiment.

In a preferred embodiment, the lens apparatus 201 can be located above the light source (not pictured) and be clear of the smart phone 100 itself. This leaves an unobstructed line of sight from the user's eye through the lens apparatus 201 and through the specula apparatus 202. The otoscope attachment 200 also leaves the touch screen 305 of the smart phone 100 unobstructed allowing the user to control the light source or any other feature of the phone 100. The otoscope attachment can be comprised of metal, plastic or any other suitable material or combination of suitable materials known to those of ordinary skill in the art.

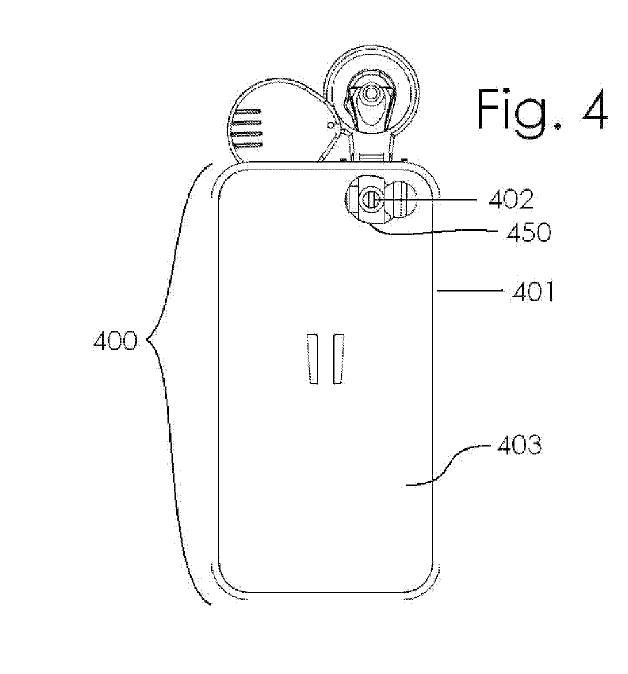

FIG. 4/8 is a rear view of an otoscope attachment 200 in an open configuration, according to an embodiment.

The otoscope attachment can comprise a phone cover 400 capable of being removably connected to the back of a smart phone (not pictured in FIG. 4/8). In a preferred embodiment, the phone cover 400 can have a slightly beveled outside edge 401 of the approximate width of the smart phone allowing it to snap over the back of the smart phone. The outside edge 401 could also comprise a lip or other similar retaining device configured to hold the smart phone within the phone cover 400. The area 403 inside the outside edges 401 of the phone cover 400 should be only slightly larger than the area of the smart phone's face so that the phone is securely held within the phone cover 400. This is important because the light source (not pictured) must be held in a specific position 402 relative to the otoscope attachment in order to properly shine light through the otoscope attachment 200. The phone cover 400 must comprise an opening 450 through which light can be transmitted from the light source to the otoscope attachment and to the patient being examined.

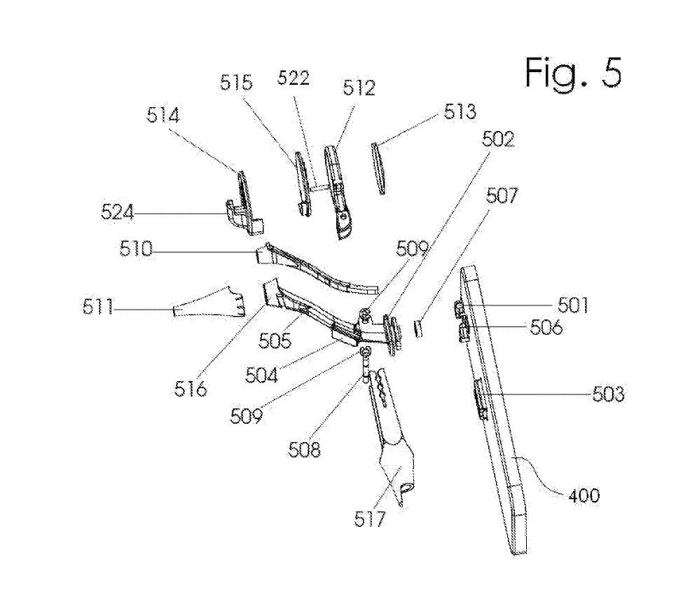

FIG. 5/8 is an exploded side perspective view of an otoscope attachment, according to an embodiment.

In this figure, the pieces comprising the otoscope attachment can be clearly viewed. The base upon which the attachment can be erected is the phone cover 400. On the front of the cover 400 is a first attachment base 501, which can be located over the hole (not pictured) in the cover 400 where the light source on the smart phone is located. A mounting arm 502 can be designed to removably connect to the first attachment base 501 when the otoscope attachment is to be placed in its open configuration. A rubber gasket 507 can be attached to the bottom of the mounting arm 502. When this gasket 507 is located between the mounting arm 502 and the smart phone, it can ensure that light from the light source is only capable of passing through the gasket and through the mounting arm 502.

The phone cover 400 can also comprise a second attachment base 503, which can accept a receiver 504 located on an otoscope arm 505. In a preferred embodiment, the receiver 504 can be removably connected to the second attachment base 503 when the otoscope attachment 200 is to be placed in its closed configuration. The cover 400 can also comprise an attachment lip 506 capable of holding a speculum retainer 516 in place against the lip 506, thus locking the otoscope arm 505 into the closed position by preventing the receiver 504 from sliding out of the second attachment base 503.

The otoscope arm 505 can be attached to the mounting arm 502 with a pin 508 and retaining clips 509. A light pipe 510 can be located within the otoscope arm 505 and stretch from the speculum retainer 516 through the mounting arm 502 to the light source. This light pipe 510 can be comprised of a fiber optic material and can be capable of carrying light from the light source to the speculum retainer 516, where it can be directed into the patient being examined. This light pipe 510 should also comprise a hollow pathway (not pictured) near the specula retainer 516 so that an unobstructed view through the retainer 516 and the speculum 511 can be maintained. In a preferred embodiment the light pipe's 510 hollow pathway is conical in shape.

The pin 508 can also be the attachment point of a magnifying lens assembly comprising a lens frame 512 configured to hold a magnifying lens 513. In a preferred embodiment, a lens cover comprising a top half 514 and a bottom half 515 can be configured to pivot around a lens frame pin 522 and protecting both sides of the magnifying lens 513. A lens cover retainer 524 can lock against the upper side of the otoscope arm 505 when the lens assembly is folded forward into the closed configuration.

Also in a preferred embodiment, a specula holder 517 can be connected to the pin 508. Several specula can be stacked within this holder 517. Having the ability to carry more than one speculum 511 at a time is an important feature because each speculum 511 must be discarded after every use in order to prevent the spread of germs.

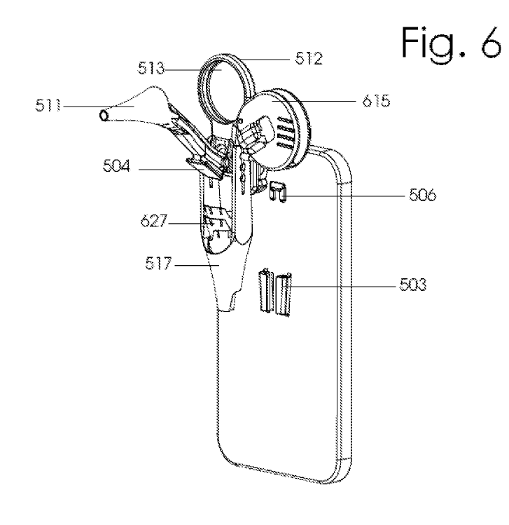

FIG. 6/8 is a perspective view of an otoscope attachment in its open configuration according to an embodiment.

This view clearly depicts the positions of the magnifying lens 513 with regard to the speculum 511 when the otoscope attachment in its open configuration and ready for use. This view also indicates how the lens cover 615 can be rotated off of the lens frame 512 when the magnifying lens 513 is to be utilized. The specula holder 517 and spare specula 627 contained within it are clearly visible in this view. Also clearly visible are the receiver 504, the attachment lip 506, and the second attachment base 503. These parts are all critical for holding the otoscope attachment in its closed configuration for both storage and portability purposes.

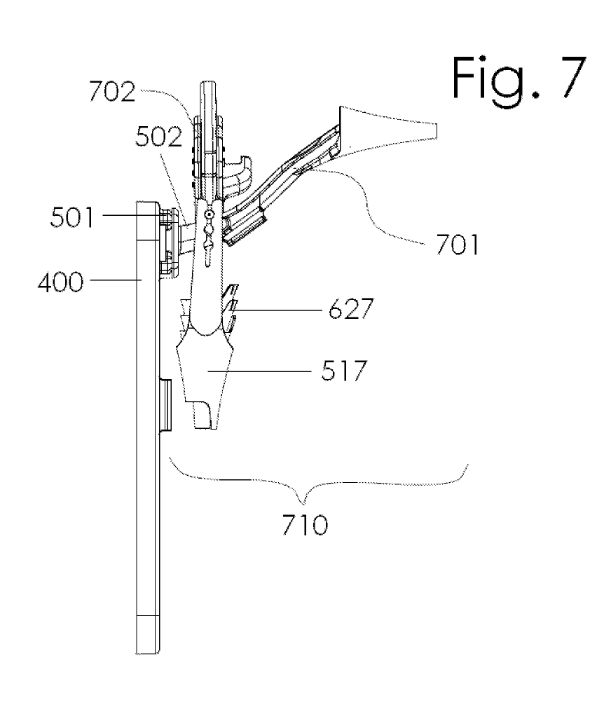

FIG. 7/8 is a side perspective view of an otoscope attachment in an open configuration, according to an embodiment.

The otoscope attachment can be viewed as being comprised of two main pieces.

The first piece is the phone cover 400, which can be formed from a single piece of plastic or comparable material. The second piece, referred to hereinafter as the “working section” 710 is an interconnected mass of parts, which comprise nearly all of the working parts of the otoscope apparatus, including the speculum assembly 701 and the lens assembly 702. The working section 710 also includes the specula holder 517 and spare specula 627. This entire working section 710 can be disconnected from the phone cover 400 by disconnecting the mounting arm 502 from the first attachment base 501. This feature is critically important to the achievement of a more compact and portable device.

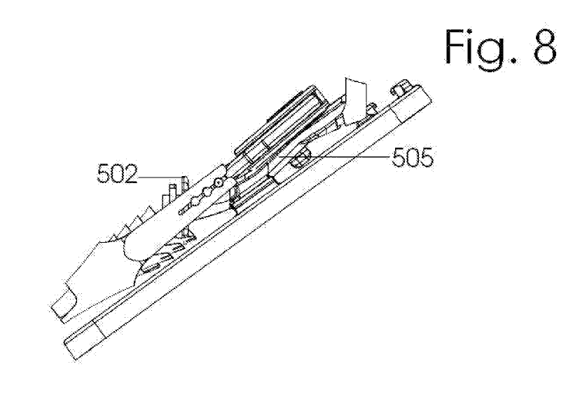

FIG. 8/8 is a side perspective view of an otoscope attachment in a closed configuration, according to an embodiment.

FIG. 8/8 depicts an important feature of the present otoscope attachment, namely its ability to be transformed into a closed configuration, which is both compact and portable. In this closed configuration, the otoscope attachment 200 is mostly flat and capable of being stored in a shirt pocket, lab coat pocket or other comparable space. As mentioned above, the key to achieving this compact shape is the ability to remove nearly all of the working parts comprising the working section 710 of the otoscope attachment 200, from the phone cover 400. Once removed this working section 710 can be folded nearly flat as depicted in FIG. 8/8. Once folded, the working section 710 can then be connected to the phone cover 400 by sliding the receiver 504 into the second attachment base 503 until the speculum receiver 516 snaps into place over the attachment lip 506. The phone cover 400 can easily be snapped off of the back of the smart phone before or after the removal of the working section 710 from the phone cover 400.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. An otoscope attachment for use with a cellular phone comprising:
a phone cover configured to removably connect to the back of a cellular phone, wherein the cellular phone comprises a light source;
a working section comprising a speculum assembly and a lens assembly, the speculum assembly further comprising a light pipe configured to transmit light from the light source through a speculum removably connected to the speculum assembly.

2. The otoscope attachment as described in claim 1 wherein, the lens assembly comprises a magnifying lens.

3. The otoscope attachment as described in claim 1 wherein, the phone cover comprises an opening over the light source.

4. The otoscope attachment as described in claim 1 wherein, the working section also comprises a specula holder.

5. The otoscope attachment as described in claim 1 wherein, the phone cover and the working section are comprised of plastic.

6. The otoscope attachment as described in claim 1 wherein, the light pipe is comprised of a fiber optic material.

7. The otoscope attachment as described in claim 1 wherein, the light pipe has a conical shape at one end.

8. An otoscope attachment for use with a cellular phone having at least one light source comprising:
a phone cover configured to removably connect to the back of a cellular phone;
a working section configured to removably connect to the phone cover, the working section further comprising a speculum assembly and a lens assembly, wherein the speculum assembly comprises a light pipe configured to transmit light from the light source through a speculum removably connected to the speculum assembly; and
the working section is configured to be folded nearly flat and reconnected to the phone cover.

9. The otoscope attachment as described in claim 8 wherein, phone cover comprises an attachment base and the speculum assembly comprises a receiver configured to be accepted by the attachment base.

10. The otoscope attachment as described in claim 1 wherein, the lens assembly comprises a magnifying lens.

11. The otoscope attachment as described in claim 8 wherein, the phone cover comprises an opening over the light source.

12. The otoscope attachment as described in claim 8 wherein, the working section also comprises a specula holder.

13. The otoscope attachment as described in claim 8 wherein, the phone cover and the working section are comprised of plastic.

14. The otoscope attachment as described in claim 8 wherein, the light pipe is comprised of a fiber optic material.

15. The otoscope attachment as described in claim 8 wherein, the light pipe has a conical shape at one end.

16. A method for using an otoscope attachment comprising:
providing a cellular phone having at least one light source, a speculum, and an otoscope attachment for use with a cellular phone having at least one light source comprising a phone cover configured to removably connect to the back of a cellular phone, a working section configured to removably connect to the phone cover, the working section further comprising a speculum assembly and a lens assembly, wherein the speculum assembly comprises a light pipe configured to transmit light from the light source through a speculum removably connected to the speculum assembly, and wherein the working section is configured to be folded nearly flat and reconnected to the phone cover;
removably connecting the phone cover to the back of the cellular phone such that the light source can transmit light through a hole in the phone cover;
connecting a speculum to the speculum assembly;
connecting the working section to the phone cover such that the light source can transmit light through the light pipe comprising the speculum assembly, through the speculum;
positioning the lens assembly so that a clear line of sight exists from the users eye, through the lens and through the speculum;
activating the light source on the cellular phone; and
using the cellular phone and otoscope attachment to examine a patient.

17. The otoscope attachment as described in claim 1 wherein the light source is a light-emitting diode.

18. The otoscope attachment as described in claim 8 wherein the light source is a light-emitting diode.

19. The otoscope attachment as described in claim 15 wherein the light source is a light-emitting diode.

20. The otoscope attachment as described in claim 8 wherein the cellular phone is a smart phone.
